(19) United States
(12) Patent Application Publication
(10) Pub. No.: US 2021/0007717 A1
(43) Pub. Date: Jan. 14, 2021

(54) DIGITAL ULTRASOUND CABLE AND ASSOCIATED DEVICES, SYSTEMS, AND METHODS

(71) Applicant: KONINKLIJKE PHILIPS N.V., EINDHOVEN (NL)

(72) Inventor: BERNARD JOSEPH SAVORD, ANDOVER, MA (US)

(21) Appl. No.: 16/969,682

(22) PCT Filed: Jan. 31, 2019
(86) PCT No.: PCT/EP2019/052310

§ 371 Date: Aug. 13, 2020

Related U.S. Application Data
(60) Provisional application No. 62/631,549, filed on Feb. 16, 2018.

(51) Int. Cl. A61B 8/00; A61B 8/08; G01S 7/52

(52) U.S. Cl. CPC A61B 8/56; A61B 8/5207; A61B 8/4444; G01S 7/5208; G01S 7/52025; G01S 7/52034; A61B 8/4494

(57) ABSTRACT

Ultrasound image devices, systems, and methods are provided. In one embodiment, a medical ultrasound imaging system (100) includes a communication link (150) including at least one data lane in communication with a host system (130); and an ultrasound imaging probe (110), comprising an ultrasound imaging component (112) configured to provide a plurality of analog ultrasound echo channel signals (160); a plurality of analog-to-digital converters, ADCs (116) coupled to the ultrasound imaging component (112), the plurality of ADCs (116) configured to generate channelized ultrasound echo data streams (162) based on the plurality of analog ultrasound echo channel signals (160); a multiplexer, MUX (118) coupled to the plurality of ADCs (116) and configured to multiplex the channelized ultrasound echo data streams (162) into a multiplexed channelized ultrasound echo data stream (164); and a communication interface (122) coupled to the MUX (118) and the communication link (150), the communication interface (122) configured to transmit a digital signal including the multiplexed channelized ultrasound echo data stream (164) to the host system (130).

### TECHNICAL FIELD

The present disclosure relates generally to ultrasound imaging and, in particular, to transporting digital ultrasound data streams from a transducer probe to a host system over a low-cost, high-speed, digital multi-lane communication link.

### BACKGROUND

Ultrasound imaging systems are widely used for medical imaging. An ultrasound imaging system typically includes a transducer probe separate from a main processing system. The transducer probe may include an array of ultrasound transducer elements. The ultrasound transducer elements send acoustic waves through a patient's body and records echoes as the acoustic waves are reflected back by the tissues and/or organs within the patient's body. The timing and/or strength of the echoes may represent the size, shape, and mass of the tissues and/or organs of the patient. Traditionally, raw analog ultrasound echo signals are passed through a cable from the transducer probe to the main processing system. In some instances, the analog ultrasound echo signals may be pre-amplified at the transducer probe before transferring to the main processing system. The main processing system processes the raw analog ultrasound echo signals to produce image signals for display.

The cable that connects the transducer probe to the main processing system can be relatively long since the main processing system is typically located on a cart and the transducer probe is required to be placed on the anatomy of interest. In addition, the cable can be complex and the size or the diameter of the cable can be large as the cable is required to carry received echo signals from each ultrasound transducer element to the main processing system. For example, the transducer probe can include an array of 128 transducers, each forming a transmit channel and a receive channel. To transfer ultrasound echo signals for each channel, the cable is required to include about 128 conductors or wires. As such, the wire-count in the cable can be high, and thus the cost of a cable can be the costliest component in an ultrasound imaging system.

One approach to reducing the wire-count in the ultrasound system cable is to include electronics in the transducer probe for analog sub-array processing or analog partial beamforming. However, analog processing can limit the quality of the signal as well as the number of simultaneous acoustic lines that can be formed.

Another approach to overcoming the limitations of analog processing is to include low-power analog-to-digital converters (ADCs) in the transducer probe, perform full beamforming digitally at the transducer probe, and transfer the beamformed signals to the main processing system using a universal serial bus (USB) cable. However, the beamforming circuitry can consume a large amount of power exceeding the thermal budget of the system. In addition, the flexibility and/or capability of the beamforming circuitry may be limited due to the small-size constraint of the transducer probe.

Still another approach to reducing the cost, the complexity, and/or the size of the cable is to migrate from analog communications to digital communications. For example, digital signals can be sent over a few wires using standard communication protocols, such as a universal serial bus (USB) standard. However, today's digital communication protocols may not have a sufficient bandwidth to support signals with high image quality and high frame rates. For example, a high-quality ultrasound imaging system may include a transducer probe with about 192 transducer elements. Each transducer element may provide an analog ultrasound echo channel signal. To transfer the analog ultrasound echo signals over a digital link, the transducer probe may include about 192 analog-to-digital converters (ADCs) for digitizing the analog ultrasound echo channel signals into digital signals. To provide a sufficient resolution for representing the original ultrasound echo signal waveforms, the transducer probe may employ high-performance ADCs that can produce digital samples with a bit-width of about 14 bits at a sampling rate of about 40 megahertz (MHz). Thus, the digital communication link between the transducer probe and the main processing system may be required to support a data transfer rate of about 107.52 gigabits per second (Gbps).

An additional approach to reducing the amount of data across the digital communication link is to perform beamforming at the transducer probe. However, beamforming at the transducer probe is typically statically configured, and thus may limit the flexibility of manipulating or processing per-channel ultrasound echo signals at the main processing system. Nonetheless, the beamformed signals may still require a high data transfer rate. For example, a 32-channel beamformed outputs with 24-bit samples at a sampling rate of about 40 MHz may require a data transfer rate of about 30.72 Gbps. Current standard low-cost interfaces, such as USB version 3.0, can only support a data transfer rate of about one-tenth of such a data transfer rate. While an optical communication link can be used to provide the necessary data transfer rate, optical communications may draw a high amount of electrical power from the system causing the system to exceed the system thermal budget.

### SUMMARY

While existing ultrasound imaging systems have proved useful for medical imaging and diagnosis, there remains a need for improved systems and techniques for reducing the costs of high-quality ultrasound imaging systems that use a large number of transducer elements to produce high-resolution images at a high frame rate. Embodiments of the present disclosure provide mechanisms for transferring per-channel digital ultrasound echo channel signals from a transducer probe to a host system. For example, the transducer probe can include a plurality of analog-to-digital converters (ADCs) coupled to an array of transducer elements. The transducer elements can emit ultrasound waves for imaging a patient's body and receive echoes as the ultrasound waves are reflected from the patient's body. The echoes are analog signals. The ADCs can generate per-channel digital ultrasound echo signals from the analog ultrasound echo channel signals received from each transducer element. The per-channel digital ultrasound echo signals represent the original analog waveforms of the analog ultrasound echo channel signals. The transducer probe can include one or more digital multiplexers (MUX) for multiplexing multiple per-channel digital ultrasound echo channel signals for transmissions over a cable assembly including multiple data lanes. The multiple data lanes can provide a data transfer rate in excess of 12 gigabits per second (Gbps). The host system can perform beamforming, signal processing, and/or image processing on the per-channel digital ultrasound echo channel signals for image display.

In one embodiment, a medical ultrasound imaging system includes a communication link including at least one data lane in communication with a host system; and an ultrasound imaging probe, comprising an ultrasound imaging component configured to provide a plurality of analog ultrasound echo channel signals; a plurality of analog-to-digital converters (ADCs) coupled to the ultrasound imaging component, the plurality of ADCs configured to generate channelized ultrasound echo data streams based on the plurality of analog ultrasound echo channel signals; a multiplexer (MUX) coupled to the plurality of ADCs and configured to multiplex the channelized ultrasound echo data streams into a multiplexed channelized ultrasound echo data stream; and a communication interface coupled to the MUX and the communication link, the communication interface configured to transmit a digital signal including the multiplexed channelized ultrasound echo data stream over the at least one data lane to the host system.

In some embodiments, the ultrasound imaging component includes an array of transducer elements, and wherein each of the plurality of ADCs is coupled to one of the transducer elements and configured to generate one channel data stream of the channelized ultrasound echo data streams based on a corresponding analog ultrasound echo channel signal. In some embodiments, the MUX comprises a first MUX coupled to a first subset of the plurality of ADCs and configured to multiplex corresponding channelized ultrasound echo data streams into a first multiplexed channelized ultrasound echo data stream; and a second MUX coupled to a second subset of the plurality of ADCs and configured to multiplex corresponding channelized ultrasound echo data streams into a second multiplexed channelized ultrasound echo data stream, wherein the communication interface is further configured to simultaneously transmit a first digital signal including the first multiplexed channelized ultrasound echo data stream over a first data lane of the communication link and a second digital signal including the second multiplexed channelized ultrasound echo data stream over a second data lane of the communication link. In some embodiments, the ultrasound imaging probe further comprises a processing component configured to determine whether a data size of the generated channelized ultrasound echo data streams exceeds a threshold associated with an image depth, and wherein the communication interface is further configured to transmit the digital signal based on the determination. In some embodiments, the ultrasound imaging probe further comprises an encoder coupled to the MUX and configured to encode the multiplexed channelized ultrasound echo data stream into an encoded data stream, and wherein the communication interface is further configured to transmit the digital signal by transmitting the digital signal including the encoded data stream over the at least one data lane to the host system. In some embodiments, the encoded data stream includes a control word indicating a start of the encoded data stream. In some embodiments, the medical ultrasound imaging system further comprises the host system comprising a communication interface coupled to the communication link and configured to receive the digital signal including the multiplexed channelized ultrasound echo data stream from the communication link; and a decoder coupled to the communication interface and configured to decode the digital signal to produce a decoded data stream, and a de-multiplexer (DeMUX) coupled to the decoder and configured to de-multiplex the multiplexed channelized ultrasound echo data stream into de-multiplexed channelized ultrasound echo data streams. In some embodiments, the communication interface of the host system further comprises a clock recovery component configured to recover a clock signal from the received digital signal for the decoding. In some embodiments, the host system further comprises a beamforming component configured to generate a beamformed signal based on the de-multiplexed channelized ultrasound echo data streams; a signal processing component coupled to the beamforming component and configured to generate an image signal based on the beamformed signal; and a display configured to display the image signal. In some embodiments, the ultrasound imaging probe further includes at least one of an analog beamforming component coupled to the ultrasound imaging component and the plurality of ADCs, the analog beamforming component configured to perform partial beamforming on the plurality of analog ultrasound echo channel signals; or a digital beamforming component coupled to the plurality of ADCs and the MUX, the digital beamforming component configured to perform partial beamforming on the channelized ultrasound echo data streams. In some embodiments, the communication interface includes a signal conditioning component configured to perform at least one of high-frequency pre-emphasis or low-frequency de-emphasis on the digital signal. In some embodiments, the communication interface includes a current mode logic (CML) component configured to generate the digital signal based on the multiplexed channelized ultrasound echo data stream. In some embodiments, the communication link further includes a plurality of twisted pairs forming a plurality of data lanes, and wherein the communication link includes a data transfer rate of at least 12 gigabits per second. In some embodiments, the medical ultrasound imaging system further comprises a coupling component configured to couple the communication link to the host system, wherein the coupling component includes a beamforming component configured to generate a beamformed signal based on the channelized ultrasound echo data streams.

In one embodiment, a method of medical ultrasound imaging includes receiving, from an ultrasound imaging component of an ultrasound imaging probe, a plurality of analog ultrasound echo channel signals; generating, via a plurality of analog-to-digital converters (ADCs) of the ultrasound imaging probe, channelized ultrasound echo data streams based on the plurality of analog ultrasound echo channel signals; multiplexing, via a multiplexer (MUX) of the ultrasound imaging probe, the channelized ultrasound echo data streams into at least one multiplexed channelized ultrasound echo data stream; and transmitting, to a host system via at least one data lane of a communication link, a digital signal including the multiplexed channelized ultrasound echo data stream.

In some embodiments, the multiplexing includes multiplexing, via a first MUX coupled to a first subset of the plurality of ADCs, corresponding channelized ultrasound echo data streams into a first multiplexed channelized ultrasound echo data stream; and multiplexing, via a second MUX coupled to a second subset of the plurality of ADCs, corresponding channelized ultrasound echo data streams into a second multiplexed channelized ultrasound echo data stream, and wherein the transmitting includes simultaneously transmitting a first digital signal over a first data lane of the communication link and a second digital signal over a second data lane of the communication link, the first digital signal including the first multiplexed channelized ultrasound echo data stream, and the second digital signal including the second multiplexed channelized ultrasound echo data stream. In some embodiments, the method further comprises determining whether a data size of the generated channelized ultrasound echo data streams exceeds a threshold associated with an image depth, wherein the transmitting includes transmitting the digital signal based on the determining. In some embodiments, the method further comprises encoding, via an encoder of the ultrasound imaging probe, the multiplexed channelized ultrasound echo data stream into an encoded data stream, wherein the transmitting includes transmitting the digital signal including the encoded data stream over the at least one data lane to the host system. In some embodiments, the method further comprises receiving, by the host system, the digital signal including the multiplexed channelized ultrasound echo data stream from the communication link; decoding, by the host system, the digital signal into a decoded data stream; and de-multiplexing, by the host system, the decoded data stream into de-multiplexed channelized ultrasound echo data streams. In some embodiments, the method further comprises generating, by the host system, a beamformed signal based on the de-multiplexed channelized ultrasound echo data streams; and generating, by the host system, an image signal based on the beamformed signal; and displaying, by the host system, the image signal.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an ultrasound imaging system, according to aspects of the present disclosure.

FIG. 2 is a schematic diagram illustrating a transducer portion of an ultrasound imaging system, according to aspects of the present disclosure.

FIG. 3 is a schematic diagram illustrating a host portion of an ultrasound imaging system, according to aspects of the present disclosure.

FIG. 4 is a frequency response diagram illustrating cable dispersions effects, according to aspects of the present disclosure.

FIG. 5 is a schematic diagram illustrating an example probe circuitry, according to aspects of the present disclosure.

FIG. 6 is a timing diagram illustrating digital transmissions over a cable, according to aspects of the present disclosure.

FIG. 7 is a timing diagram illustrating transmissions over a digital multi-lane communication link, according to aspects of the present disclosure.

FIG. 8 is a schematic diagram illustrating an example successive approximation analog-to-digital converter (ADC), according to aspects of the present disclosure.

FIG. 9 is a flow diagram of a medical ultrasound imaging method, according to aspects of the present disclosure.

FIG. 10 is a schematic diagram of an ultrasound imaging system, according to aspects of the present disclosure.

### DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

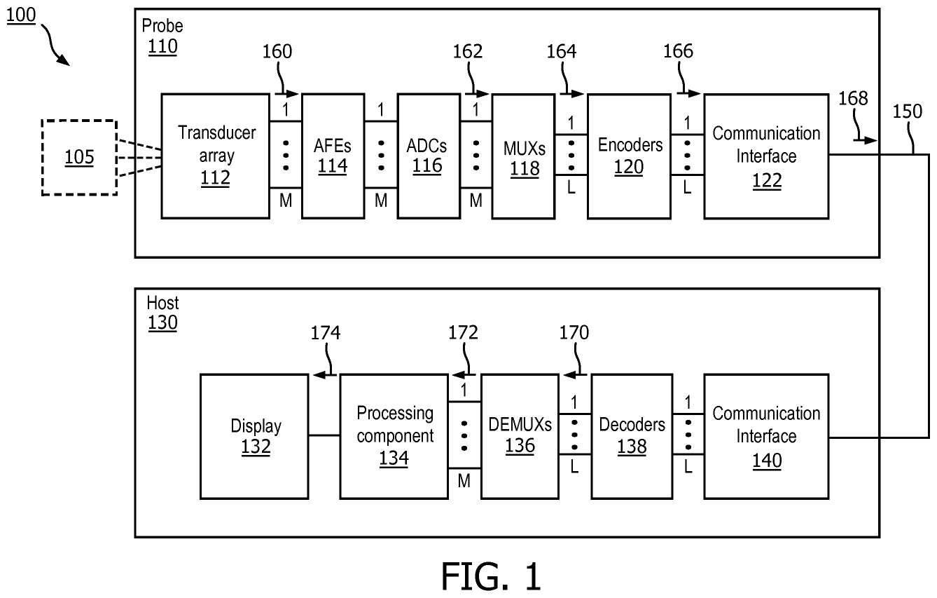

FIG. 1 is a schematic diagram of an ultrasound imaging system 100, according to aspects of the present disclosure. The system 100 is used for scanning an area or volume of a patient's body. The system 100 includes an ultrasound imaging probe 110 in communication with a host 130 over a communication interface or link 150. At a high level, the probe 110 emits ultrasound waves towards an anatomical object 105 (e.g., a patient's body) and receives echoes that are reflected from the object 105. The probe 110 transmits per-channel echo signals digitally over the link 150 to the host 130 for process and image display. The probe 110 may be in any suitable form for imaging various body parts of a patient while positioned inside or outside of the patient's body. For example, the probe 110 may be in the form of a catheter, a transesophageal echocardiography (TEE) probe, an endo-cavity probe, a handheld ultrasound scanner, or a patch-based ultrasound device.

The probe 110 includes a transducer array 112, a plurality of analog frontends (AFEs) 114, a plurality of analog-to-digital converters (ADCs) 116, a plurality of multiplexers (MUXs) 118, a plurality of encoders 120, and a communication interface 122. The host 130 includes a display unit 132, a processing component 134, a plurality of de-multiplexers (DEMUXs) 136, a plurality of decoders 138, and a communication interface 140.

The transducer array 112 emits ultrasound signals towards the object 105 and receives echo signals reflected from the object 105 back to the transducer array 112. The transducer array 112 may include acoustic elements arranged in a one-dimensional (1D) array or in a two-dimensional (2D) array. The acoustic elements may be referred to as transducer elements. Each transducer element can emit ultrasound waves towards the object 105 and can receive echoes as the ultrasound waves are reflected back from the object 105. For example, the transducer array 112 can include M plurality of transducer elements producing M plurality of analog ultrasound echo channel signals 160. In some embodiments, M can be about 2, 16, 64, 128, 192, or greater than 192.

The AFEs 114 are coupled to the transducer array 112 via M signal lines. Each AFE 114 may be coupled to one transducer element in the transducer array 112. The AFEs 114 may include circuitry configured to provide high voltage excitations and gain controls. The high voltage excitations can trigger ultrasound wave emissions at the transducer elements. The gain controls can provide low-noise pre-amplification to the received echoes.

The ADCs 116 are coupled to the AFEs 114 via M signal lines. Each ADC 116 may be coupled to one AFE 114 and configured to convert a corresponding analog ultrasound echo channel signal 160 into a digital ultrasound echo channel signal 162. In some embodiments, the ADCs 116 are successive approximation type ADCs. The successive approximation ADC architecture can provide high-performance and lower-power consumption, and thus may keep total power dissipation of the probe 110 to be within a thermal budget of the probe 110. The ADCs 116 can produce M plurality of digital ultrasound echo channel signals 162. Each digital ultrasound echo channel signal 162 includes digital samples representing the waveforms of a corresponding analog ultrasound echo channel signal. The M plurality of digital ultrasound echo channel signals 162 may be referred to as per-channel ultrasound echo data streams or channelized ultrasound echo data streams.

The MUXs 118 are coupled to the ADCs 116 via M signal lines. Each MUX 118 may be coupled to a subset of the ADCs 116 via a corresponding subset of signal lines and configured to multiplex a corresponding subset of the channelized ultrasound echo data streams 162. As an example, the ADCs 116 are grouped into L subsets. Thus, the probe 110 may include L plurality of MUXs 118 producing L plurality of multiplexed data streams 164. The MUXs 118 are digital MUXs and can be implemented using a combination of hardware components and software components.

The encoders 120 are coupled to the MUXs 118 via L signal lines. Each encoder 120 may be coupled to one MUX 118 and configured to encode a corresponding multiplexed data stream 164 into an encoded data stream 166. The encoders 120 can be implemented using a combination of hardware components and software components. In some embodiments, the encoders 120 may implement an 8b10b encoding algorithm as described in U.S. Pat. No. 4,486,739. The 8b10b encoding maps an 8-bit input data unit into 10-bit output symbols. The 8b10b encoding maximizes the number of bit-transitions in the encoded data stream 164 and can provide a minimal direct current (DC) component. The encoders 120 can produce L plurality of encoded data streams 166.

The communication interface 122 is coupled to the encoders 120 via L signal lines. The communication interface 122 is configured to transmit the L encoded data streams 166 to the host 130 via the communication link 150. The communication interface 122 may include a combination of hardware components and software components configured to generate digital signals 168 carrying the encoded data streams 166 for transmission over the communication link 150. The communication link 150 may include L data lanes for transferring the digital signals 168 to the host 130, as described in greater detail herein.

The host 130 may be any suitable computing and display device, such as a workstation, a personal computer (PC), a laptop, a tablet, a mobile phone, or a patient monitor. In some embodiments, the host 130 may be located on a moveable cart. At the host 130, the communication interface 140 may receive the digital signals 168 from the communication link 150. The communication interface 140 may include a combination of hardware components and software components. The communication interface may be substantially similar to the communication interface 122 in the probe 110.

The decoders 138 are coupled to the communication interface 140 via L signal lines. Each decoder 138 is configured to receive a digital signal 168 from one of the data lanes and perform decoding on the digital signal 168 to recover a decoded data stream 170. The decoding may be performed according to the encoding algorithm (e.g., according to the 8b10b encoding) used by the encoders 120 at the probe 110. The decoders 138 can be implemented using a combination of hardware components and software components. The decoders 138 can produce L plurality of decoded data streams 170.

The DEMUXs 136 are coupled to the decoders 138 via L signal lines. Each DEMUX 136 may be coupled to one decoder 138 and configured to de-multiplex a corresponding decoded data stream 170 into a plurality of data streams 172 corresponding to the per-channel ultrasound echo data streams at the output of the ADCs 116. The DEMUXs 136 can produce M de-multiplexed ultrasound echo data streams 172.

The processing component 134 is coupled to the DEMUXs 136 via M signal lines. The processing component 134 may include a central processing unit (CPU), a digital signal processor (DSP), a graphical processing unit (GPU), an application-specific integrated circuit (ASIC), a controller, a field-programmable gate array (FPGA), another hardware device, a firmware device, or any combination thereof. The processing component 134 may also be implemented as a combination of computing devices, e.g., a combination of a DSP and a microprocessor, a GPU and a microprocessor, a plurality of microprocessors, one or more microprocessors in conjunction with a DSP core, or any other such configuration. The processing component 134 can be configured to generate image signals 174 from the de-multiplexed ultrasound echo data streams 172 and/or perform image processing and image analysis for various diagnostic modalities.

The display unit 132 is coupled to the processing component 134. The display unit 132 may include a monitor, a touch-screen, or any suitable display. The display unit 132 is configured to display images and/or diagnostic results processed by the processing component 134. The host 130 may further include a keyboard, a mouse, or any suitable user-input components configured to receive user inputs for controlling the system 100.

While FIG. 1 is described in the context of transferring detected ultrasound echo data from the probe 110 to the host 130 for display, the host 130 can generate controls for configuring the probe 110, for example, the excitations of the transducer elements at the transducer array 112, as described in greater detail herein.

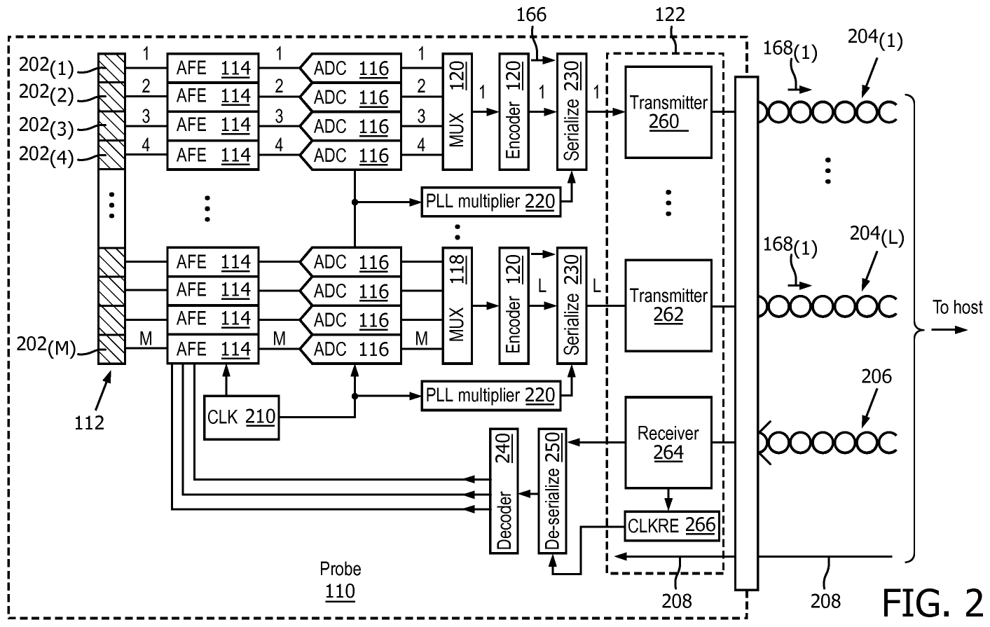

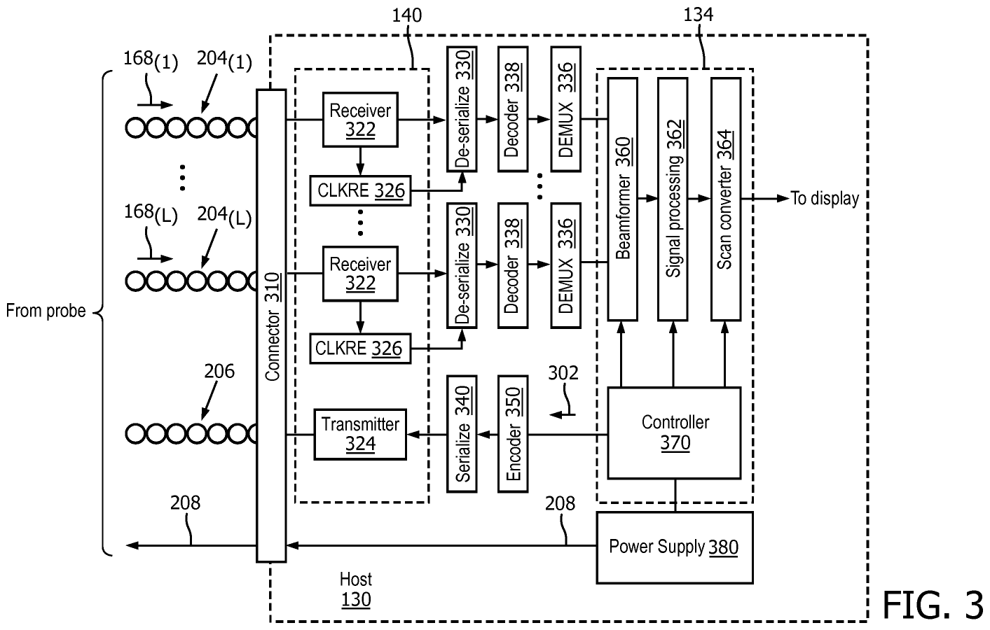

FIGS. 2 and 3 collectively provide a more detailed view of the system 100 including transmission paths from the probe 110 to the host 130 and from the host 130 to the probe 110. FIG. 2 is a schematic diagram illustrating a transducer portion of the ultrasound imaging system 100, according to aspects of the present disclosure. FIG. 2 provides a more detailed view of the internal components in the probe 110. FIG. 3 is a schematic diagram illustrating a host portion of the ultrasound imaging system 100, according to aspects of the present disclosure. FIG. 3 provides a more detailed view of the internal components in the host 130.

As shown in FIG. 2, the probe 110 further includes a clock (CLK) 210, L plurality of phase-locked loop (PLL) multipliers 220, L plurality of serializing components 230, a decoder 240, a de-serializing component 250, and a connector 270. The communication interface 122 may include L plurality of transmitters 260, a receiver 264, and a clock recovery (CLKRE) component 266. The connector 270 couples the communication interface 122 to the communication link 150.

As shown in FIG. 3, the host 130 further includes a connector 310, L plurality of de-serializing components 330, a serializing component 340, an encoder 350 and a power supply 380. The communication interface 140 may include L receivers 322, a transmitter 324, and L plurality of CLKRE components 326. The processing component 134 may include a beamformer 360, a signal processing component 362, a scan converter 364, and a controller 370. The power supply 380 may provide power to the host 130 and to the probe 110.

As shown in FIGS. 2 and 3, the communication link 150 includes L plurality of data lanes 204 (e.g., shown as 204(1) to 204(L)) for transmitting signals to the host 130, a data lane 206 for receiving signals from the host 130, and a power line 208 for receiving power from the power supply 380 in the host 130. For example, the communication link 150 may be a cable including conductors, wires, or low-cost twisted pairs that form the data lanes 204 and 206 and the power line 208.

The transmission path from the probe 110 to the host 130 may begin at the transducer array 112 shown in FIG. 2. As shown, the transducer array 112 includes M plurality of transducer elements 202, each connecting to an AFE 114. The transducer elements 202 are shown as 202₍₁₎ to 202_{(M)}.

The CLK 210 may function as a master clock in the probe 110. The CLK 210 may provide a clock signal to the AFEs 114 and the ADCs 116. The ADCs 116 may be grouped into groups of four ADCs 116, and thus L may be M/4. In some other embodiments, the ADCs 116 may be grouped into groups of 2, 8, or more than 8. Each MUX 118 may be coupled to one group of ADCs 116.

Each serializing component 230 is coupled to the output of an encoder 120. As described above, the encoders 120 may encode an 8-bit input data unit into 10-bit output symbols. The serializing component 230 may convert the output symbols (e.g., the encoded data streams 166) into a bit stream for transmission.

Each PLL multiplier 220 is coupled to the CLK 210 and a serializing component 230. The PLL multipliers 220 are configured to convert the frequency of the clock signal into a suitable frequency for operating the serializing components 230. As an example, the CLK 210 may provide a clock frequency of about 40 MHz and the ADCs 116 may be 12-bit ADCs. When the encoders 120 produce 10 bits of output for every 8 bits of input, the serializing component 230 is required to operate at a data rate of about 2.4 gigabits per second (Gbps). Thus, each PLL multiplier 220 may convert the 40 MHz clock signal into a 2.4 GHz clock signal for operating a corresponding serializing component 230.

Each transmitter 260 is coupled to one serializing component 230. The transmitter 260 may include circuitry for driving the communication link 150. The transmitters 260 may receive the encoded bit streams 166 and generate digital signals 168 carrying the encoded bit streams 166 for transmission over the data lanes 204. The digital signals 168 are shown as 168(1) to 168_{(L)}, each corresponding to one of the encoders 120. The transmissions of the digital signals 168₍₁₎ to 168_{(L)} may occur simultaneously over corresponding data lanes 204₍₁₎ to 204_{(L)}. In some embodiments, the transmitters 260 may implement a current mode logic (CML) physical layer for the transmissions, as described in greater detail herein.

Referring to the example described above, where the CLK 210 runs at 40 MHz, the ADCs 116 provides 12-bit samples, and the ADCs 116 are grouped into groups of 4. When the transducer array 112 includes 128 (e.g., M=128) transducer elements 202, the communication link 150 may include 32 data lanes, each with a data transfer rate of about 2.4 Gbps. Thus, the communication link 150 may provide a data transfer rate of about 76.8 Gbps.

As shown in FIG. 3, at the host, the receivers 322 may receive the digital signals 168 carrying the encoded, multiplexed channelized ultrasound echo data streams via the data lanes 204 of the communication link 150. Each CLKRE component 326 is coupled to a receiver 322 and a de-serializing component 330. The CLKRE component 326 is configured to recover a clock signal from the received digital signal 168 and provide the clock signal to a corresponding receiver 322 and de-serializing component 330. The receivers 322 may recover the bit stream transmitted by the probe110 over corresponding data lanes 204. The de-serializing components 330 may convert the recovered bit streams into symbols based on the bit-size of the output symbols produced by the encoder 120 at the probe 110. For example, when the encoder 120 implements the 8b10b encoding, the de-serializing components 330 may form data in units of 10 bits. Thus, each de-serializing component 330 may produce a stream of 10-bits data and provide the data stream to a corresponding decoder 138 for decoding.

The decoders 138 and the DEMUXs 136 may operate as described above to recover the channelized ultrasound echo data streams generated at the probe 110. The beamformer 360 is configured to apply timing delays to the ultrasound echo channel data streams 172 to align the timings of the different channels and may sum the time-aligned ultrasound echo channel data streams to produce beamformed signals. The signal processing component 362 is configured to perform filtering and/or quadrature demodulation to condition the beamformed signals. The signal processing component 362 may perform analytic detection and/or any image processing techniques on the conditioned signals to produce image signals 174. The scan converter 364 is configured to convert the image signals 174 into images for display, for example, on the display unit 132. The controller 370 may control the operations of the beamformer 360, the signal processing component 362, and/or the scan converter 364.

The transmission path from the host 130 to the probe 110 may begin at the controller 370 of the host 130 shown in FIG. 3. The controller 370 may further generate control data 302 for operating the transducer elements 202 at the transducer array 112, for example, for ultrasound wave emissions. At the host, the encoder 350 is coupled to the controller. The encoder 350 may be substantially similar to the encoder 120. For example, the encoder 350 may encode the control data using the same encoding algorithm (e.g., the 8b10b encoding algorithm) as the encoder 120. The serializing component 340 is coupled to the transmitter 324. The serializing component 340 may be substantially similar to the serializing component 230. For example, the serializing component 340 may convert the encoded control data stream into a bit stream. The transmitter 324 may be substantially similar to the transmitters 260. For example, the transmitter 324 may generate a digital signal carrying the encoded control data bit stream for transmission over the data lane 206.

At the probe 110, the receiver 264 may receive the digital signal carrying the encoded control data bit stream from the host 130. The CLKRE component 266 is coupled to the receiver 264. The receiver 264 may be substantially similar to the receiver 322. The CLKRE component 266 may be substantially similar to the CLKRE components 326. For example, the CLKRE component 266 may recover a clock signal from the received digital signal and the receiver 264 may recover the bit streams transmitted by the host 130 from the receive signals. The de-serializing component 250 is coupled to the receiver 264 and the decoder 240. The de-serializing component 250 may be substantially similar to the de-serializing components 330. The decoder 240 may be substantially similar to the decoders 138. For example, the de-serializing component 250 may convert the bit stream into a data stream and the decoder 240 may perform decoding to recover the control data transmitted by the host 130. The decoder 240 is coupled to the AFEs 114. For example, the control data may include excitation information for trigger ultrasound wave emissions at the transducer elements 202.

As can be seen, the inclusions of the ADCs 116 at the probe 110 allows the transfer of per-channel digital ultrasound echo data channels from the probe 110 to the host 130 for maximum processing flexibility. The use of the MUXs 118 and the parallel multi-lane communication link 150 provides a high-speed data transfer rate that is at an order of magnitude higher than currently available standard digital communication protocols (e.g., USB 3.0).

In some embodiments, the required bandwidth over the communication link 150 can be reduced by including an analog sub-array processor at the probe 110 between the AFEs 114 and the ADCs 116. The sub-array processor can perform partial beamforming to combine a subset of the analog ultrasound echo channel signals 160. The partial beamforming can further reduce the number of signal lines (e.g., the data lanes 204) required in the transmission path between the probe 110 and the host 130 or reduce the required data transfer data for each data lane 204. The full beamforming can be performed at the host 130.

In some embodiments, the required bandwidth over the communication link 150 can be reduced by including a digital partial beamformer at the probe 110 between the ADCs 116 and the MUXs 118. The digital partial beamformer can perform partial beamforming to combine a subset of the digital ultrasound echo channel data streams 162. In such embodiments, the MUXs 118 can multiplex the partial beamformed ultrasound echo data streams for encoding by the encoders 120. Similar to the analog partial beaforming, the digital partial beamforming can further reduce the number of signal lines (e.g., the data lanes 204) required in the transmission path between the probe 110 and the host 130 or reduce the required data transfer data for each data lane 204. The full beamforming can be performed at the host 130.

In some embodiments, the required bandwidth over the communication link 150 can be reduced by including both the analog partial beamformer and the digital partial beamformer at the probe 110 as described above.

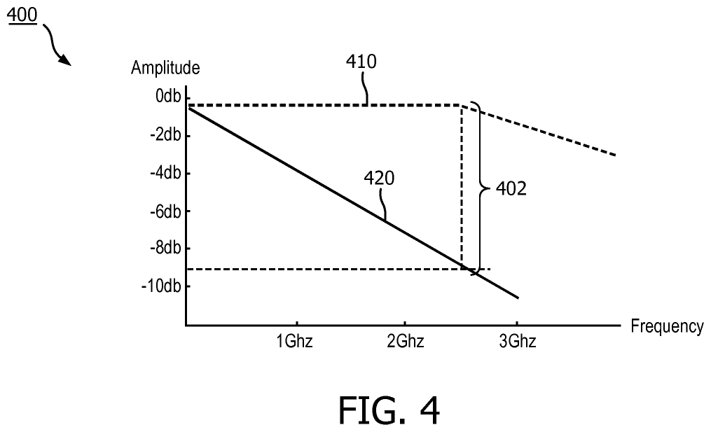

FIG. 4 is a frequency response diagram 400 illustrating cable dispersions effects, according to aspects of the present disclosure. In FIG. 4, the x-axis represents frequency in units of gigahertz (GHz) and the y-axis represents amplitude in units of decibels (dB). The curve 420 shows cable loss in a twisted pairs (e.g., used for the data lanes 204 and 206) as a function of frequencies. As can be seen, the cable loss increases as the frequency increases. The losses can prevent high-speed transmission.

In order to provide high-speed transmission, a transmitter (e.g., the transmitters 260 and 324) may perform high-frequency pre-emphasis. The curve 410 shows the cable frequency response with high-frequency pre-emphasis. As can be seen, the high-frequency pre-emphasis can provide a flat response, for example, up to about 2.6 GHz with a gain 402 of about 9 dB. In some embodiments, the transmitters 260 and 324 can be configured to implement high-frequency pre-emphasis as shown by the curve 410 to enable high data rate transmissions (e.g., at about 2.4 Gbps) over the data lanes 204 and 206. Mechanisms for implementing high-frequency pre-emphasis are described in greater detail herein.

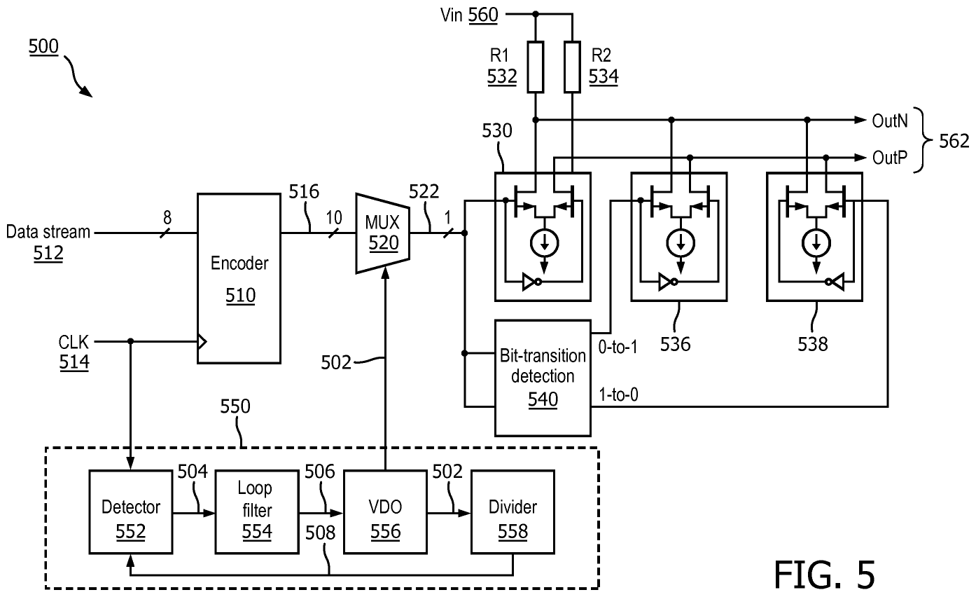

FIG. 5 is a schematic diagram illustrating example probe circuitry 500, according to aspects of the present disclosure. The circuitry 500 can be implemented by the probe 110. The circuitry 500 includes an encoder 510, a MUX 520, CML components 530, 536, and 538, a bit-transition detector 540, and a PLL frequency multiplier 550. The encoder 510 may correspond to the encoders 120 and 350. The MUX 520 may correspond to the serializing components 230 and 340. The PLL frequency multiplier 550 may correspond to the PLL multipliers 220. The CML components 530, 536, and 538 and the bit-transition detector 540 may be implemented by the transmitters correspond to the transmitters 260 and 324.

As shown, the encoder 510 receives an input data stream 512 and a clock signal 514. The input data stream 512 may correspond to a multiplexed ultrasound echo data stream 164 output by a MUX 118. The encoder 510 encodes every 8 bits from the data stream 512 into 10-bit symbols forming an encoded data stream 516 (e.g., the encoded data streams 166). The MUX 520 serializes the data stream 516 into a bit stream 522.

Referring to the example described above, where the CLK 210 runs at 40 MHz, the ADCs 116 provides 12-bit samples, and the ADCs 116 are grouped into groups of 4. Thus, the bit stream 522 may be a 2.4 Gbps serial bit stream. The clock signal 514 may be a 240 MHz clock signal. The PLL frequency multiplier 550 may generate a clock signal 502 at 2.4 Gbps for operating the MUX 520.

The PLL frequency multiplier 550 includes a detector 552, a loop filter 554, a voltage controlled oscillator (VCO), and a divider 558. The detector 552 may be a phase detector or a frequency detector. The detector 552 may compare the frequencies or the phases of the clock signal 502 and the signal 508 output by the divider 558 and generate a difference signal 504 including the frequency differences or the phase differences. The loop filter 554 may filter out any undesirable signal components from the difference signal 504 to produce a filtered signal 506. The VCO 556 may generate a clock signal (e.g., at 2.4 GHz) and apply adjustments based on the filtered signal 506. The VCO 556 may provide the clock signal 502 to the MUX 520. The VCO 556 may also provide the clock signal 502 to the divider 558. The divider 558 may divide the frequency of the clock signal 502 by a factor of 10 to provide a 240 MHz signal 508, which is fed back to the detector 552 for comparisons and adjustments.

The CML components 530, 536, and 538 include differential voltage to current converters. As described above, the 8b10b encoding maximizes the number of bit transitions, for example, 1-to-0 transitions or 0-to-1 transitions. The maximizing of the bit transitions can 0facilitate clock recovery at a receiver (e.g., the receivers 264 and 322). The bit-transition detector 540 can detect bit transitions from 0 to 1 or from 1 to 0. The CML component 530 generates a differential signal pair 562 (shown as OutP and OutN) for transmissions of the bit stream 522. The CML component 536 may amplify each bit after a 0-to-1 bit-transition and the CML component 538 may amplify each bit after a 1-to-0 bit transitions. The CML components 530, 536, and 538 may be coupled to a voltage rail 560 (shown as Vin) via a resistor 532 (shown as R1) and a resistor 534 (shown as R2). The resistors 532 and 534 may have a resistance of about 50 ohms, which may be matched to the impedance of a cable forming the communication link 150 to absorb cable reflections.

While FIG. 5 is described in the context of high-frequency pre-emphasis, the circuitry 500 may be alternatively configured to provide low-frequency de-emphasis. For example, instead of implementing bit-transition detection using the bit-transition detector 540, an edge detection filter (e.g., a finite impulse response (FIR)) may be used for low-frequency de-emphasis.

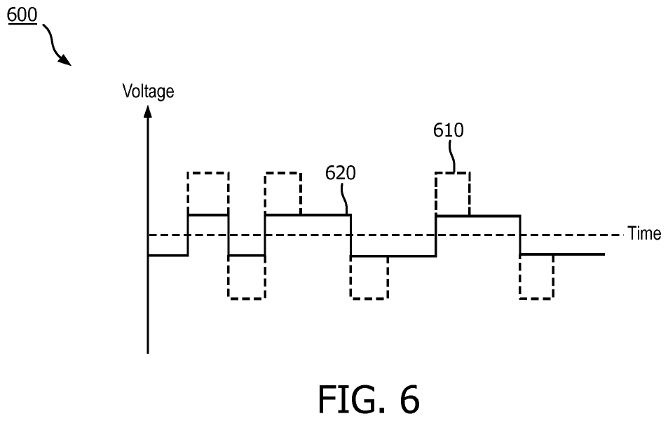

FIG. 6 is a timing diagram 600 illustrating a digital transmission over a cable, according to aspects of the present disclosure. In FIG. 6, the x-axis may represent time in some constant units and the y-axis may represent voltage levels in some constant units. FIG. 6 shows the transmissions of a serial bit stream 610 and a serial bit stream 620. The serial bit stream 620 may correspond to the bit stream 522 without the high-frequency pre-emphasis described above with respect to FIGS. 4 and 5. The serial bit stream 610 may correspond to the bit stream 522 after the high-frequency pre-emphasis.

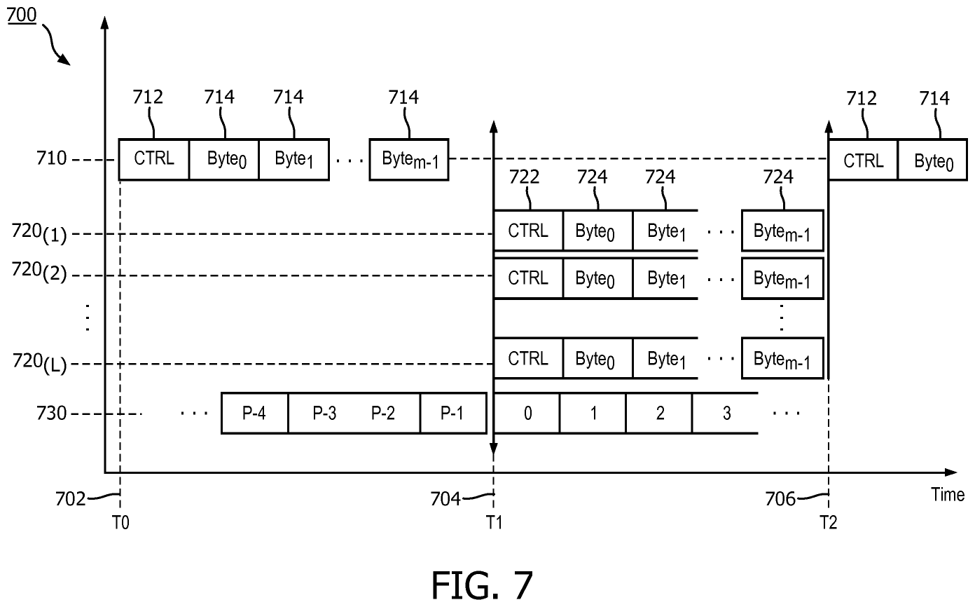

FIG. 7 is a timing diagram 700 illustrating transmissions over the digital multi-lane communication link 150, according to aspects of the present disclosure. In FIG. 7, the x-axis represents time in some constant units and the y-axis represents transmission activities over the link 150. For example, the probe 110 may employ a counter 730 to facilitate the triggering or excitations of the transducer elements 202 and the transmission of the encoded ultrasound echo channel data streams 166 over the link 150. The counter 730 may begin with a counter value of 0 and count up to P, where P is a positive integer.

At time 702, denoted as TO, the host 130 may send a control data stream 710 (e.g., the control data 302) to the probe 110 over the data lane 206 for configuring the transducer array 112. The control data stream 710 may begin with a control (CTRL) code 712 followed by n bytes of control data 714. The CTRL code 712 may be a unique identifier, such as a K.28.1 code sequence. The CTRL code 712 indicates the start of a data stream transmission.

At time 704, denoted as T1, upon receiving n bytes of control data 714 and the counter 730 counts up to P-1, the transducer elements 202 may be triggered to emit ultrasound waves, for example, towards the object 105. The value n may be a positive integer and may be predetermined. The transducer elements 202 may receive echoes of the ultrasound waves reflected by the object 105.

Upon receiving m bytes of ultrasound echo data (e.g., the encoded channelized ultrasound echo data streams 166) from each channel, the probe 110 may send the channelized ultrasound echo data streams 720 over the L data lanes 204 to the host 130. The value m may be a positive integer and may be determined based on a desired image acquisition depth. Each echo data stream 720₍ᵢ₎ may be send over a corresponding data lane 204₍ᵢ₎, where i may vary from 1 to L. As shown, the probe 110 may begin each transmission in a channel (i) with a CTRL code 722 (e.g., K.28.1 code) followed by m bytes of ultrasound echo data 724.

At time 706, denoted as T2, after transferring m bytes of ultrasound echo data 724 for each channel, the host 130 may send a next control data stream 710 for a next acquisition interval. In some embodiments, the host 130 may begin the transmission of a control data stream for a next acquisition interval at the same as the echo data streams 720 are received. While the counter 730 is described as a count-up counter, the counter 730 may be alternatively configured to be a count-down counter.

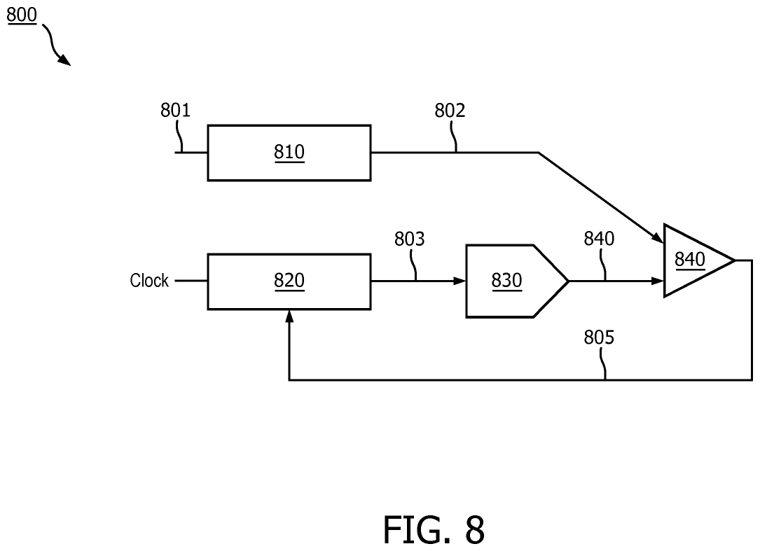

FIG. 8 is a schematic diagram illustrating an example successive approximation ADC 800, according to aspects of the present disclosure. The ADC 800 may correspond to an ADC 116 at the probe 110. The ADC 800 includes a track and hold component 810, a successive approximation register (SAR) logic component 820, a digital-to-analog converter (DAC) 830, and a comparator 840. The ADC 800 may receive an analog signal 801 (e.g., the analog ultrasound echo channel signal). The track and hold component 810 samples the analog signal 801 and holds the value for a period of time producing a sampled signal 802. The comparator 840 compares the sampled signal 802 to an output signal 804 of the DAC 830 and outputs a result signal 806 to the SAR logic component 820. The SAR logic component 820 provides an approximation digital code 803 for the analog input signal 801 to the DAC 830. The DAC 830 provides an analog signal 804 of the approximation code to the comparator 840.

In an embodiment, the SAR logic component 820 initializes the approximation code 803 with the most significant bit (MSB) set to a digital 1. The code 803 is fed into the DAC 830. The DAC 830 produces an analog signal 804 equivalent of the digital code 803 and feeds the analog signal 804 into the comparator 840. When the analog input signal 802 exceeds the voltage level of the analog signal 804, the SAR logic component 820 resets the MSB to a digital 0. Otherwise, the MSB remains with a digital value of 1. The SAR logic component 820 repeats the process of testing each bit from the MSB to the LSB. When the SAR logic component 820 completes testing all bits, the resulting code 803 is the digital approximation of the voltage of the analog input signal 801.

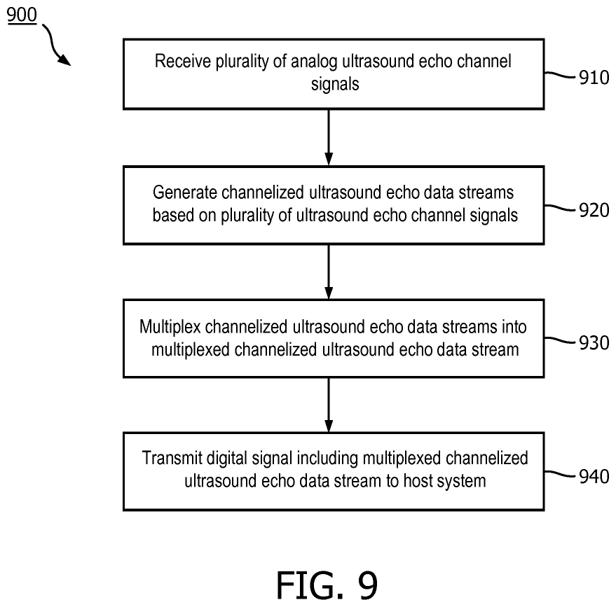

FIG. 9 is a flow diagram of a medical ultrasound imaging method 900, according to aspects of the present disclosure. Steps of the method 900 can be executed by a computing device (e.g., a processor, processing circuit, and/or other suitable component) of an ultrasound imaging probe, such as the probe 110, and/or or a host such as the host 130. The method 900 may employ similar mechanisms for transferring data between the probe 110 and the host 130 as described above with respect to FIGS. 2-8. As illustrated, the method 900 includes a number of enumerated steps, but embodiments of the method 900 may include additional steps before, after, and in between the enumerated steps. In some embodiments, one or more of the enumerated steps may be omitted or performed in a different order.

At step 910, the method 900 includes receiving a plurality of analog ultrasound echo channel signals (e.g., the analog ultrasound echo channel signals) from an ultrasound imaging component (e.g., the transducer array 112) of an ultrasound imaging probe (e.g., the probe 110).

At step 920, the method 900 includes generating channelized ultrasound echo data streams (e.g., the channelized ultrasound data streams 162) based on the plurality of analog ultrasound echo channel signals by a plurality of ADCs (e.g., the ADCs 116) of the ultrasound imaging probe.

At step 930, the method 900 includes multiplexing the channelized ultrasound echo data streams into at least one multiplexed channelized ultrasound echo data stream (e.g., the multiplexed data stream 164) by a MUX (e.g., the MUXs 118).

At step 940, the method 900 includes transmitting a digital signal (e.g., the digital signals 168) including the multiplexed channelized ultrasound echo data stream to a host system (e.g., the host).

In an embodiment, the ultrasound imaging component may include an array of transducer elements (e.g., the transducer elements 202). Each of the plurality of ADCs is coupled to one of the transducer elements and configured to generate one channel data stream of the channelized ultrasound echo data streams based on a corresponding analog ultrasound echo channel signal.

In an embodiment, the multiplexing can employ a first MUX and a second MUX. The first MUX can be coupled to one subset of the plurality of ADCs. The second MUX can be coupled to another subset of the plurality of ADCs. The first MUX can multiplex channelized ultrasound echo data streams from the first subset of ADCs into a first multiplexed channelized ultrasound echo data stream. The second MUX can multiplex a corresponding channelized ultrasound echo data streams from the second subset of ADCs into a second multiplexed channelized ultrasound echo data stream. The transmitting can include simultaneously transmitting a first digital signal (e.g., the digital signal 168(1)) over a first data lane (e.g., the data lane 204(1)) of the communication link and a second digital signal (e.g., the digital signal 168(L)) over a second data lane (e.g., the data lane 204(L)) of the communication link, the first digital signal including the first multiplexed channelized ultrasound echo data stream, and the second digital signal including the second multiplexed channelized ultrasound echo data stream.

In an embodiment, the method 900 can further include determining whether a data size (e.g., m) of the generated channelized ultrasound echo data streams exceeds a threshold associated with an image depth. The transmitting may be based on the determination.

In an embodiment, the method 900 can further include encoding the multiplexed channelized ultrasound echo data stream into an encoded data stream (e.g., the encoded data streams 166) using by an encoder (e.g., the encoders 120) of the ultrasound imaging probe. The transmitting can include transmitting the digital signal including the encoded data stream over the at least one data lane to the host system.

In an embodiment, the host system can receive the digital signal including the multiplexed channelized ultrasound echo data stream from the communication link. The host system can decode the digital signal into a decoded data stream (e.g., the decoded data streams 170). The host system can de-multiplex the decoded data stream into de-multiplexed channelized ultrasound echo data streams (e.g., the data streams 172). The host system can generate a beamformed signal based on the de-multiplexed channelized ultrasound echo data streams. The host system can generate an image signal based on the beamformed signal. The host system can display the image signal on a display (e.g., the display unit 132).

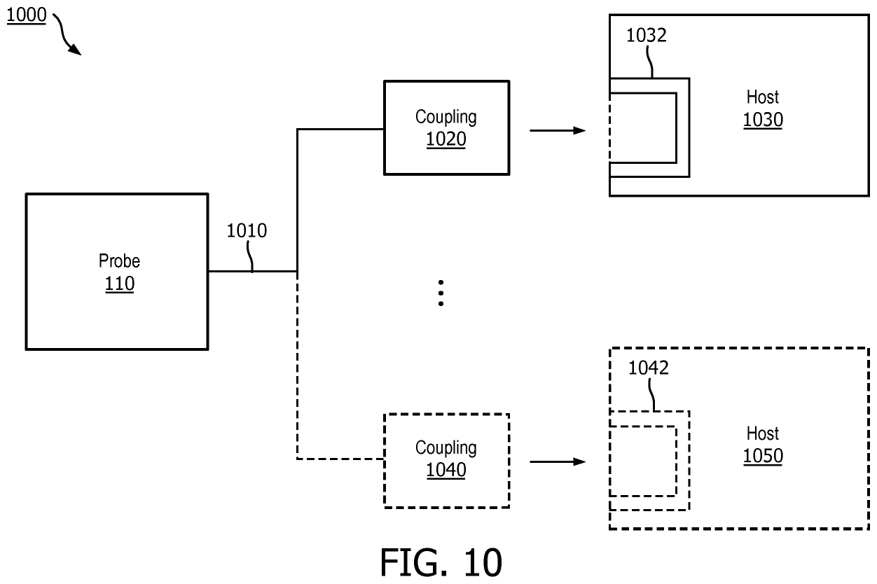

FIG. 10 is a schematic diagram of an ultrasound imaging system 1000, according to aspects of the present disclosure. The system 1000 is substantially similar to the system 100, but may implement at least some host processing functions described above at a coupling component 1020 that can be connected to a host 1030. The system 1000 includes the probe 110 coupled to a cable 1010. The coupling component 1020 is located at the end of the cable 1010 opposite the probe 110. The cable 1010 may include a plurality of twisted pairs forming data lanes similar to the data lanes 204 and 206.

The coupling component 1020 can be a connector, an adapter, or a dongle. The coupling component 1020 can include at least some of the components of the host 130 described above with respect to FIGS. 1 and 3. For example, the communication interface 140, the decoders 138, the DEMUXs 136, and the processing component 134 can reside in the coupling component 1020. The coupling component 1020 can be connected to or plugged into the host 1030 via a digital interface 1032 for display and user controls. The processing component 134 can perform at least some beamforming and the host 1030 can perform further beamforming and/or any suitable signal processing and/or image processing functions. The inclusion of a beamformer at the coupling component 1020 can remove the thermal power associated with beamforming from the probe 110 and can allow a reduction in data rate so that the coupling component 1020 can be plugged into a standard digital interface (e.g., the interface 1032), such as a USB interface. In some embodiments, the coupling component 1020 can include a wireless communication component configured to wirelessly communicate ultrasound echo data and/or user controls with the host 1030.

As shown, the system 1000 can be configured for a different host 1050 by replacing the coupling component 1020 with a coupling component 1040. The host 1050 may be substantially similar to the host 1030. For example, the host 1050 may be a workstation, a laptop, a tablet, or a mobile phone. The coupling component 1040 may be substantially similar to the coupling component 1020. For example, the coupling component 1040 can include similar functional components (e.g., the decoders 138, the DEMUXs 136, and the processing component 134) as the coupling component 1020. However, the coupling component 1040 and the host 1050 may be coupled to a digital interface 1042 different than the digital interface 1032. For example, the digital interface 1042 may be a USB 2.0/3.0/3.1 interface and the digital interface 1032 may be an Ethernet interface.

Aspects of the present disclosure can provide several benefits. For example, the use of multiple wires within a cable bundle to send multiple high speed serial data streams in parallel can provide an increased digital bandwidth using a low cost flexible cable. The use of 8b10b encoding enable clock recovery from a received data bit stream using a PLL, and this may compensate for data skew between the parallel wires. In addition, the 8b10b encoding can provide for handshakes (e.g., the CTRL codes 712 and 722) with minimal overhead. The use of high-frequency pre-emphasis or low-frequency de-emphasis can compensate for the frequency dependent loss in low-cost cable. The use of CML interface with terminations at both transmit and receive ends can minimize effects of cable reflections. The use of a counter (e.g., the counter 730) to time acquisition intervals can provide a steady continuous ultrasound data stream without the need for large data buffering or complex hand-shaking, and thus can reduce latency. The transfer of per-channel digital ultrasound echo signals directly to the host 130 can provide flexibility in the processing of the signals, and thus can provide a high image quality and frame rate. The integrating of cable communications directly with the ADCs can maintain low power consumption. The use of low-power successive approximation type ADCs (e.g., the ADC 800) can keep the total power dissipation within the thermal budget of the probe 110.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A medical ultrasound imaging system, comprising:
a communication link including at least one data lane in communication with a host system; and
an ultrasound imaging probe, comprising:
an ultrasound imaging component configured to provide a plurality of analog ultrasound echo channel signals;
a plurality of analog-to-digital converters (ADCs) coupled to the ultrasound imaging component, the plurality of ADCs configured to generate channelized ultrasound echo data streams based on the plurality of analog ultrasound echo channel signals;
a multiplexer (MUX) coupled to the plurality of ADCs and configured to multiplex the channelized ultrasound echo data streams into a multiplexed channelized ultrasound echo data stream; and
a communication interface coupled to the MUX and the communication link, the communication interface configured to transmit a digital signal including the multiplexed channelized ultrasound echo data stream over the at least one data lane to the host system.

2. The medical ultrasound imaging system of claim 1, wherein the ultrasound imaging component includes an array of transducer elements, and wherein each of the plurality of ADCs is coupled to one of the transducer elements and configured to generate one channel data stream of the channelized ultrasound echo data streams based on a corresponding analog ultrasound echo channel signal.

3. The medical ultrasound imaging system of claim 1, wherein the MUX comprises:
a first MUX coupled to a first subset of the plurality of ADCs and configured to multiplex corresponding channelized ultrasound echo data streams into a first multiplexed channelized ultrasound echo data stream; and
a second MUX coupled to a second subset of the plurality of ADCs and configured to multiplex corresponding channelized ultrasound echo data streams into a second multiplexed channelized ultrasound echo data stream,
wherein the communication interface is further configured to simultaneously transmit a first digital signal including the first multiplexed channelized ultrasound echo data stream over a first data lane of the communication link and a second digital signal including the second multiplexed channelized ultrasound echo data stream over a second data lane of the communication link.

4. The medical ultrasound imaging system of claim 1, wherein the ultrasound imaging probe further comprises:
a processing component configured to determine whether a data size of the generated channelized ultrasound echo data streams exceeds a threshold associated with an image depth, and
wherein the communication interface is further configured to transmit the digital signal based on the determination.

5. The medical ultrasound imaging system of claim 1, wherein the ultrasound imaging probe further comprises:
an encoder coupled to the MUX and configured to encode the multiplexed channelized ultrasound echo data stream into an encoded data stream, and
wherein the communication interface is further configured to transmit the digital signal by transmitting the digital signal including the encoded data stream over the at least one data lane to the host system.

6. The medical ultrasound imaging system of claim 5, wherein the encoded data stream includes a control word indicating a start of the encoded data stream.

7. The medical ultrasound imaging system of claim 5, further comprising:
the host system comprising:
a communication interface coupled to the communication link and configured to receive the digital signal including the multiplexed channelized ultrasound echo data stream from the communication link; and
a decoder coupled to the communication interface and configured to decode the digital signal to produce a decoded data stream, and
a de-multiplexer (DeMUX) coupled to the decoder and configured to de-multiplex the multiplexed channelized ultrasound echo data stream into de-multiplexed channelized ultrasound echo data streams.

8. The medical ultrasound imaging system of claim 7, wherein the communication interface of the host system further comprises:
a clock recovery component configured to recover a clock signal from the received digital signal for the decoding.

9. The medical ultrasound imaging system of claim 7, wherein the host system further comprises:
a beamforming component configured to generate a beamformed signal based on the de-multiplexed channelized ultrasound echo data streams;
a signal processing component coupled to the beamforming component and configured to generate an image signal based on the beamformed signal; and
a display configured to display the image signal.

10. The medical ultrasound imaging system of claim 1, wherein the ultrasound imaging probe further includes at least one of:
an analog beamforming component coupled to the ultrasound imaging component and the plurality of ADCs, the analog beamforming component configured to perform partial beamforming on the plurality of analog ultrasound echo channel signals; or
a digital beamforming component coupled to the plurality of ADCs and the MUX, the digital beamforming component configured to perform partial beamforming on the channelized ultrasound echo data streams.

11. The medical ultrasound imaging system of claim 1, wherein the communication interface includes:
a signal conditioning component configured to perform at least one of high-frequency pre-emphasis or low-frequency de-emphasis on the digital signal.

12. The medical ultrasound imaging system of claim 1, wherein the communication interface includes:
a current mode logic (CML) component configured to generate the digital signal based on the multiplexed channelized ultrasound echo data stream.

13. The medical ultrasound imaging system of claim 1, wherein the communication link further includes a plurality of twisted pairs forming a plurality of data lanes, and wherein the communication link includes a data transfer rate of at least 12 gigabits per second.

14. The medical ultrasound imaging system of claim 1, further comprising:
a coupling component configured to couple the communication link to the host system,
wherein the coupling component includes a beamforming component configured to generate a beamformed signal based on the channelized ultrasound echo data streams.

15. A method of medical ultrasound imaging, comprising:
receiving, from an ultrasound imaging component of an ultrasound imaging probe, a plurality of analog ultrasound echo channel signals;
generating, via a plurality of analog-to-digital converters (ADCs) of the ultrasound imaging probe, channelized ultrasound echo data streams based on the plurality of analog ultrasound echo channel signals;
multiplexing, via a multiplexer (MUX) of the ultrasound imaging probe, the channelized ultrasound echo data streams into at least one multiplexed channelized ultrasound echo data stream; and
transmitting, to a host system via at least one data lane of a communication link, a digital signal including the multiplexed channelized ultrasound echo data stream.

16. The method of claim 15, wherein the multiplexing includes:
multiplexing, via a first MUX coupled to a first subset of the plurality of ADCs, corresponding channelized ultrasound echo data streams into a first multiplexed channelized ultrasound echo data stream; and
multiplexing, via a second MUX coupled to a second subset of the plurality of ADCs, corresponding channelized ultrasound echo data streams into a second multiplexed channelized ultrasound echo data stream, and
wherein the transmitting includes simultaneously transmitting a first digital signal over a first data lane of the communication link and a second digital signal over a second data lane of the communication link, the first digital signal including the first multiplexed channelized ultrasound echo data stream, and the second digital signal including the second multiplexed channelized ultrasound echo data stream.

17. The method of claim 15, further comprising:
determining whether a data size of the generated channelized ultrasound echo data streams exceeds a threshold associated with an image depth,
wherein the transmitting includes transmitting the digital signal based on the determining.

18. The method of claim 15, further comprising:
encoding, via an encoder of the ultrasound imaging probe, the multiplexed channelized ultrasound echo data stream into an encoded data stream,
wherein the transmitting includes transmitting the digital signal including the encoded data stream over the at least one data lane to the host system.

19. The method of claim 18, further comprising:
receiving, by the host system, the digital signal including the multiplexed channelized ultrasound echo data stream from the communication link;
decoding, by the host system, the digital signal into a decoded data stream; and
de-multiplexing, by the host system, the decoded data stream into de-multiplexed channelized ultrasound echo data streams.

20. The method of claim 19, further comprising:
generating, by the host system, a beamformed signal based on the de-multiplexed channelized ultrasound echo data streams;
generating, by the host system, an image signal based on the beamformed signal; and
displaying, by the host system, the image signal.
